(19) United States
(12) Patent Application Publication
(10) Pub. No.: US 2024/0221902 A1
(43) Pub. Date: Jul. 4, 2024

(54) GENERATION OF PERSONALIZED RECOMMENDATION TO ENSURE SAFE CONSUMPTION OF FOODS ITEMS

(71) Applicant: SONY GROUP CORPORATION, TOKYO (JP)

(72) Inventor: HODA SAYYADINEJAD, SAN DIEGO, CA (US)

(21) Appl. No.: 18/147,193

(22) Filed: Dec. 28, 2022

(51) Int. Cl. G16H 20/60; G16H 10/65

(52) U.S. Cl. CPC G16H 20/60; G16H 10/65; G06Q 30/0625

(57) ABSTRACT

An electronic device and method for personalized recommendation for safe consumption of foods items is disclosed. The electronic device collects information associated with a user of the electronic device and determines a food item that the user intends to consume. The determination is performed based on the collected information. The electronic device queries the database to determine whether the database includes a record corresponding to the determined food item. The database is for food items that cause food allergies or food intolerances to a user. The electronic device generates a recommendation associated with the food item based on whether the database includes the record and controls the display to render the recommendation. The recommendation indicates whether the food item is safe for the user to consume in light of the food allergies or the food intolerances.

### CROSS-REFERENCE TO RELATED APPLICATIONS/INCORPORATION BY REFERENCE

None

### FIELD

Various embodiments of the disclosure relate to personalized recommendations. More specifically, various embodiments of the disclosure relate to an electronic device and method for generation of personalized recommendations to ensure safe consumption of foods items.

### BACKGROUND

Dietary limitations are now more crucial than ever as more individuals experience health problems as a result of their diet. Food allergies or food intolerances may be the root of some of the most prevalent health problems. A food allergy is an unfavorable health effect brought on by a specific immunological reaction that occurs frequently following exposure to a particular meal. When a particular meal or food item cannot be digested by the body, a food intolerance may result. One of the most common and well-known food intolerances is a sensitivity to lactose. Dietary habits and preferences, as well as food products, have developed over time. With so many food alternatives available, it may be difficult for a person to identify every ingredient in a food item and recall all elements that may cause food allergy or intolerance. Many people experience an allergic reaction or other health problems after consuming a meal that they thought was safe.

Limitations and disadvantages of conventional and traditional approaches will become apparent to one of skill in the art, through comparison of described systems with some aspects of the present disclosure, as set forth in the remainder of the present application and with reference to the drawings.

### SUMMARY

An electronic device and method of generation of personalized recommendations to ensure safe consumption of foods items is provided substantially as shown in, and/or described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram that illustrates an exemplary network environment for generation of personalized recommendations to ensure safe consumption of foods items, in accordance with an embodiment of the disclosure.

FIG. 2 is a block diagram that illustrates an exemplary electronic device of FIG. 1, in accordance with an embodiment of the disclosure.

FIGS. 3A and 3B are diagrams that illustrates an exemplary scenario for generation of personalized recommendations to ensure safe consumption of foods items, in accordance with an embodiment of the disclosure.

FIGS. 4A and 4B are diagrams that illustrate an exemplary scenario for generation of personalized recommendations to ensure safe consumption of foods items based on user's location, in accordance with an embodiment of the disclosure.

FIG. 5 is a diagram that illustrates an exemplary scenario for collection of health information associated with a user of an electronic device, in accordance with an embodiment of the disclosure.

FIG. 6 is a diagram that illustrates an exemplary scenario for generation of personalized recommendations to ensure safe consumption of foods items, in accordance with an embodiment of the disclosure.

FIG. 7 is a flowchart that illustrates operations of an exemplary method for generation of personalized recommendations to ensure safe consumption of foods items, in accordance with an embodiment of the disclosure.

### DETAILED DESCRIPTION

The following described implementation may be found in the electronic device and method for generation of personalized recommendations to ensure safe consumption of foods items. Exemplary aspects of the disclosure may provide an electronic device (for example, a wearable device, a smartphone, a personal computer, a laptop, a server, and the like) that may execute operations for generation of personalized recommendations to ensure safe consumption of foods items. The electronic device may collect information associated with a user of the electronic device. The electronic device may determine a food item that the user may intend to consume, based on the collected information. The electronic device may query a database to determine whether the database includes a record corresponding to the determined food item. The electronic device may generate a recommendation associated with the food item, based on whether the database includes the record. The recommendation may indicate whether the food item is safe for the user to consume in light of the food allergies. The electronic device may control the display to render the recommendation.

Dietary restrictions have become increasingly significant as more individuals experience health problems linked to their diet. Some of the most common health problems are caused by food allergies or food intolerances. A food allergy is an undesirable health outcome that results from a particular immune response that happens repeatedly after exposure to a certain food. A food intolerance may occur when the body is unable to digest a specific food or food ingredient. Lactose intolerance is one of the most prevalent and well-known dietary intolerances. Dietary habits and choices have evolved over time, as have food items. With wide variety of food options, it may be difficult for a person to identify every ingredient in a food item and remember all ingredients that may cause food allergy or food intolerance to the person. Many times, a person may experience discomfort or an allergic response after eating a meal that they thought was safe.

The present disclosure provides a method to generate personalized recommendations that ensure safe consumption of foods items. The disclosed electronic device may store a database of food items that the user may be allergic or intolerant to. The database may be created based on past health problems or discomforts that the user may have faced after consuming certain food items. In case the user wishes to consume a food item, the electronic device may allow the user to input a name or an identifier of the food item. Upon receiving the input, the electronic device may check if the food item in included in a record of the database of food items. Since a food item may go by various names within various cultures, locations, or shops, the database may include various name variations of the food item in the form of tags. If the food item is mentioned in the database of food items, then the user may be requested or recommended to not consume the food item. Additional information may be shown to the user to inform about the allergen in the food item or an ingredient in the food item that causes a food intolerance, a severity of reaction that the user may experience after consuming the food item, or a medicine that the user may take to mitigate the severity of reaction. The user may not be required to remember the food items that cause a food allergy or a food intolerance to the user and the user may make informed decision regarding consumption of any food item.

The electronic device of the present disclosure may also determine a severity of reaction that a food item may cause to a person. Past medical conditions or current medical conditions of the user may be considered in the determination of the severity of reaction. If the severity of reaction on consumption of the food item is determined to be a severe reaction, then the user may not be recommended to consume the food item. However, if the severity of reaction on consumption of the food item is determined to be a moderate or mild reaction, then the user may be recommended to consume the food item. The determination of the severity for a food item may be decided based on statistical information about a type of medical intervention that people may have required in past after consumption of the food item. For example, if 5% of the people required hospitalization after experiencing symptoms of lactose intolerance, then the severity for milk-based food items may be determined as a moderate or mild reaction. In some cases, the electronic device may provide preventive medications that the user may consume to mitigate the symptoms of a food allergy or a food intolerance associated with the food item.

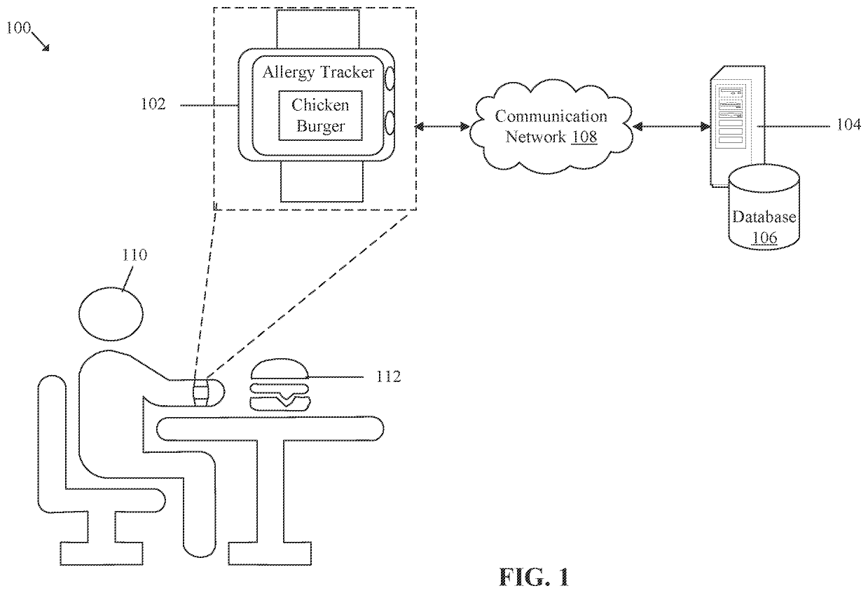

FIG. 1 is a block diagram that illustrates an exemplary network environment for generation of personalized recommendations to ensure safe consumption of foods items, in accordance with an embodiment of the disclosure. With reference to FIG. 1, there is shown a network environment 100. The network environment 100 may include an electronic device 102, a server 104, a database 106, and a communication network 108. The electronic device 102 and the server 104 may be communicatively coupled to one another, via the communication network 108. In FIG. 1, there is further shown a user 110 who may be associated with the electronic device 102. The server 104 may host the database 106. Alternatively, the electronic device 102 may store the database 106.

The electronic device 102 may include suitable logic, circuitry, interfaces, and/or code that may be configured to collect information associated with a user (such as the user 110) of the electronic device 102 and may determine a food item 112 that the user 110 intends to consume. The electronic device 102 may query a database such as the database 106 to determine whether it is safe for the user 110 to consume the determined food item 112. For such a determination, the electronic device 102 may query the database 106 to determine whether the database 106 includes a record corresponding to the determined food item 112. If the record for the food item 112 is determined to exist in the database 106, then it may indicate that the food item 112 or another food item that includes the food item 112 as an ingredient may have caused a food allergy or a food intolerance to the user 110 in the past. Thus, the electronic device 102 may identify the food item 112 as an unsafe food item and may render a recommendation that prompts the user 110 to not consume the determined food item 112.

In FIG. 1, the electronic device 102 is shown to be a wearable smart watch. However, the disclosure may not be limited to a smart watch implementation of the electronic device 102. Other example implementations of the electronic device may include, but are not limited to, a laptop, a personal computer, a smartphone, a display tablet, a head-mounted display, and the like.

The server 104 may include suitable logic, circuitry, and interfaces, and/or code that may be configured to generate a recommendation associated with the food item 112. The recommendation may indicate whether the food item 112 is safe for the user 110 to consume in light of the food allergies or the food intolerances. In one or more embodiments, the server 104 may store the database of food items that cause food allergies to the user 110 and may execute at least one operation associated with the electronic device 102. The server 104 may execute operations through web applications, cloud applications, HTTP requests, repository operations, file transfer, and the like. Other example implementations of the server 104 may include, but are not limited to, a database server, a file server, a web server, a media server, an application server, a mainframe server, or a cloud computing server.

In at least one embodiment, the server 104 may be implemented as a plurality of distributed cloud-based resources by use of several technologies that are well known to those ordinarily skilled in the art. A person with ordinary skill in the art will understand that the scope of the disclosure may not be limited to the implementation of the server 104 and the electronic device 102 as two separate entities. In certain embodiments, the functionalities of the server 104 may be incorporated in its entirety or at least partially in the electronic device 102, without a departure from the scope of the disclosure.

The database 106 may include suitable logic, interfaces, and/or code that may be configured to store the database of food items that may cause food allergies to the user 110. The database 106 may be stored or cached on a device, such as a server (e.g., the server 104) or the electronic device 102. The device storing the database 106 may be configured to query the database 106 to determine whether the database 106 includes the record corresponding to the determined food item 112. In response, the device that stores the database 106 may generate the recommendation associated with the food item 112 based on whether the database 106 includes the record.

In some embodiments, the database 106 may be hosted on a plurality of servers stored at same or different locations. The operations of the database 106 may be executed using hardware, including a processor, a microprocessor (e.g., to perform or control performance of one or more operations), a field-programmable gate array (FPGA), or an application-specific integrated circuit (ASIC). In some other instances, the database 106 may be implemented using software.

The communication network 108 may include a communication medium through which the electronic device 102 and the server 104 may communicate with one another. The communication network 108 may include one of a wired connection or a wireless connection. Examples of the communication network 108 may include, but are not limited to, the Internet, a cloud network, a Cellular or Wireless Mobile Network (such as Long-Term Evolution and 5th Generation (5G) New Radio (NR)), a Wireless Fidelity (Wi-Fi) network, a Personal Area Network (PAN), a Local Area Network (LAN), or a Metropolitan Area Network (MAN). Various devices in the network environment 100 may be configured to connect to the communication network 108 in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols.

In some embodiments, the network environment 100 may include a machine learning (ML) model on the electronic device 102 or the server 104. The ML model may be a classifier that may be trained to identify a relationship between inputs, such as features in a training dataset and output labels (such as recommendations associated with the food item 112). The ML model may be defined by its hyper-parameters, for example, number of weights, cost function, input size, number of layers, and the like. After several epochs of training on features in the training dataset, the ML model may be trained to output a prediction/classification result for a set of inputs. The prediction result may be indicative of a class label for each input of the set of inputs.

The food item 112 may be an edible substance in raw, cooked, or process form that the user 110 may be consume. Examples of the food item 112 may include, but is not limited to, a chicken burger, a fried chicken burger, pasta, pizza, hamburger, and beverages. The food item 112 may be associated with some information such as, a name of the food item (such as a tomato pizza), a type of food item (such as fast food0, and a list of ingredient (such as, flour, cheese, tomato, and the like).

The ML model may include electronic data, which may be implemented as, for example, a software component of an application executable on the electronic device 102. The ML model may rely on libraries, external scripts, or other logic/instructions for execution on a processing device, such as the server 104. The ML model may include code and routines configured to enable a computing device such as the server 104 to perform one or more operations such as, providing recommendations to the user 110. Additionally, or alternatively, the ML model may be implemented using hardware including a processor, a microprocessor (e.g., to perform or control performance of one or more operations), a field-programmable gate array (FPGA), or an application-specific integrated circuit (ASIC). Alternatively, in some embodiments, the ML model may be implemented using a combination of hardware and software.

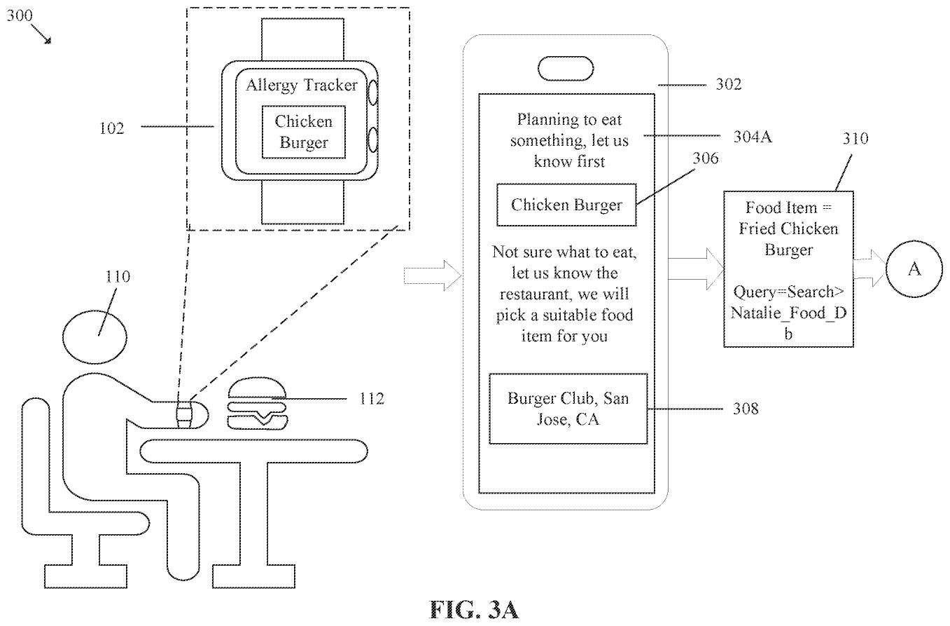

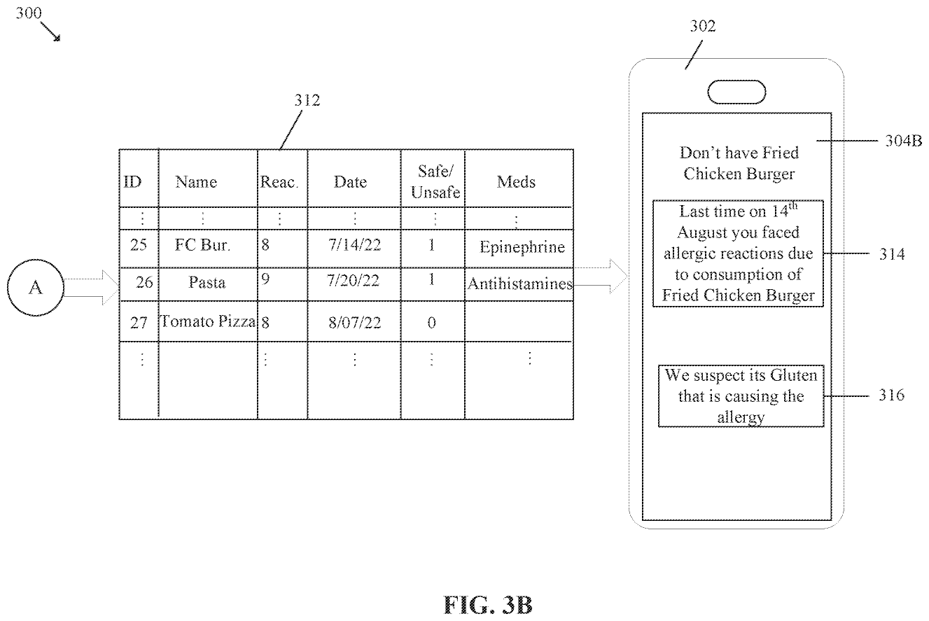

In operation, the electronic device 102 may be configured to collect information associated with the user 110 of the electronic device 102. In accordance with an embodiment, the collected information may include dietary preferences of the user 110, a current location of the user 110, or an input from the user 110 via a user interface of the electronic device 102. The input may include, for example, a text associated with the food item 112, an image of the food item 112, a video of the food item 112, an audio associated with the food item 112, and the like. The text may describe the food item 112 and the audio may include a voice description of the food item 112. Additionally, or alternatively, the collected information may include details related to the food item 112 that the user 110 intends to consume. As an example, the collected information may include a name of the food item 112 (such as a fried chicken burger), a type of food item (such as gluten free fast food), and an ingredient list for the food item (e.g., gluten free flour, chicken, eggs, cheese, and the like). Further details related to the collection of information of the food item 112 are provided, for example, in FIG. 3A.

The electronic device 102 may be configured to determine the food item 112 that the user 110 may intend to consume, based on the collected information. For example, if the collected information includes the image of the food item 112, then the image may be provided as the input to a trained ML model that may identify a name of the food item 112, a type of the food item 112, an ingredient list, and the like. Alternatively, if the collected information includes a location of the user 110, then the location may be searched in a location database to identify if the location is user's home location or a commercial location that sells food items. In case the location is a commercial location that sells food items, the electronic device 102 (or the server 104) may analyze a menu of food items that the commercial location offers and past purchases (or the dietary preferences) of the user 110 to determine the food item 112 that the user 110 is likely to order and consume. Details related to determination of the food item 112 are further provided, for example, in FIG. 3A.

The electronic device 102 may be configured to query the database 106 to determine whether the database 106 includes a record corresponding to the determined food item 112. The name or an identifier of the food item 112 may be searched in the database 106 to determine whether the record of the food item 112 exists or not.

The database 106 may include details of all the food items that the user 110 may be allergic or intolerant to. The database 106 may be created or updated based on past health problems or discomforts that the user 110 may have faced after consuming certain food items. Typically, a food item may go by various names within various cultures, locations, or shops. For example, a croissant is a type of pastry, but many people or even many stores may simply refer it as a French pastry. In some cases, trademarks or brandings may be used with food item names to make the food look different or distinctive. In many cases, one or more ingredients in a food item (e.g., a dish, a drink, etc.) may be responsible for causing an allergic reaction or a food intolerance. If such ingredients (e.g., flour with gluten) are typically used in food items of a type (e.g., pastry, cake, bread, etc.), then the database 106 may include a list of name variations of the food item 112 in the form of tags and a type or category to which the food item 112 belongs to. During search, information related to the food item 112 may be compared with the list of name variations and the type or category of the food item 112.

If the record(s) for the food item 112 exists in the database 106, then the electronic may retrieve the corresponding record(s) to determine whether the determined food item 112 is safe or unsafe to consume for the user 110. The electronic device 102 may be further configured to generate a recommendation associated with the food item 112 based on whether the database 106 includes the record(s). The recommendation may indicate whether the food item 112 is safe for the user 110 to consume in light of the food allergies or the food intolerances. For example, in case the record(s) indicates that the determined food item 112 is unsafe, then the food item 112 may not be recommended to the user 110 for consumption. A notification or a prompt may be rendered via the electronic device 102 to inform the user 110 about the safety and potential health problems associated with the consumption of the food item 112. Details related to the recommendation associated with the food item 112 are provided, for example, in FIG. 3B.

The electronic device 102 may be configured to control the display to render the recommendation. In some cases, the rendered recommendation may also include preventive medications that the user 110 may consume to mitigate effects of a food allergy or a food intolerance associated with the food item 112. Further, the rendered recommendation may indicate one or more food ingredients used in the food item 112 that the user 110 may be allergic to. The user 110 may request a restaurant or store to remove such ingredients or replace such ingredients with safe options while preparing the determined food item 112. For example, the user 110 may request a restaurant to replace cow milk with almond or oats milk. The rendered recommendation may include optional ingredients that the user 110 may consume and may request the restaurant or the store to include when preparing the food item 112. Thus, the electronic device 102 may enable the user 110 to make informed decisions related to consumption of food items at any time, without requiring the user 110 to memorize all of the food items that the user 110 must avoid. Details related to the rendering of the recommendation are provided, for example, in FIG. 3B.

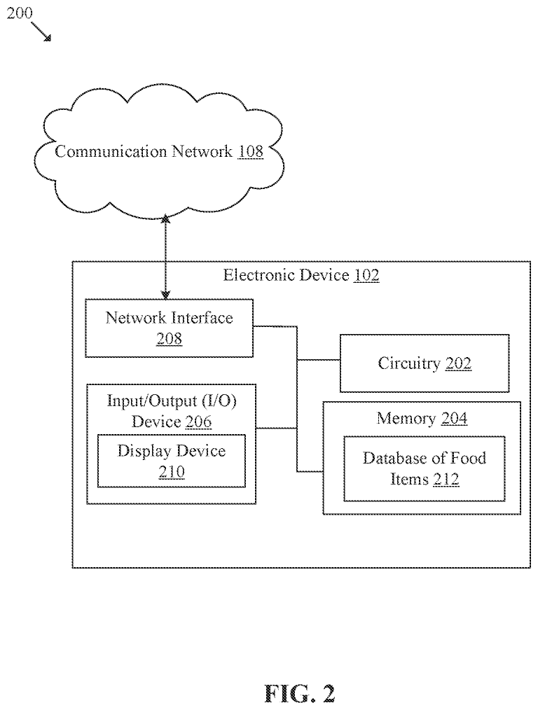

FIG. 2 is a block diagram that illustrates an exemplary electronic device of FIG. 1, in accordance with an embodiment of the disclosure. FIG. 2 is explained in conjunction with elements from FIG. 1. With reference to FIG. 2, there is shown the electronic device 102. The electronic device 102 may include circuitry 202, a memory 204, an input/output (I/O) device 206, and a network interface 208. The memory 204 may include a database of food items 212 and the input/output (I/O) device 206 may include a display device 210.

The circuitry 202 may include suitable logic, circuitry, and/or interfaces that may be configured to execute program instructions associated with different operations to be executed by the electronic device 102. The circuitry 202 may include one or more processing units, which may be implemented as a separate processor. In an embodiment, the one or more processing units may be implemented as an integrated processor or a cluster of processors that perform the functions of the one or more specialized processing units, collectively. The circuitry 202 may be implemented based on a number of processor technologies known in the art. Examples of implementations of the circuitry 202 may be an X86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other control circuits.

The memory 204 may include suitable logic, circuitry, interfaces, and/or code that may be configured to store one or more instructions to be executed by the circuitry 202. The memory 204 may be configured to the database of food items 212. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, interfaces, and/or code that may be configured to receive an input and provide an output based on the received input. For example, the I/O device 206 may receive a first user input indicative of the collected information. The I/O device 206 may be further configured to display the recommendation associated with the food item. The I/O device 206 may include the display device 210. Examples of the I/O device 206 may include, but are not limited to, a touch screen, a keyboard, a mouse, a joystick, a microphone, or a speaker.

The network interface 208 may include suitable logic, circuitry, interfaces, and/or code that may be configured to facilitate communication between the electronic device 102 and the server 104 via the communication network 108. The network interface 208 may be implemented by use of various known technologies to support wired or wireless communication of the electronic device 102 with the communication network. The network interface 208 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 208 may be configured to communicate via wireless communication with networks, such as the Internet, an Intranet, a wireless network, a cellular telephone network, a wireless local area network (LAN), or a metropolitan area network (MAN). The wireless communication may be configured to use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), 5th Generation (5G) New Radio (NR), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VOIP), light fidelity (Li-Fi), Worldwide Interoperability for Microwave Access (Wi-MAX), a protocol for email, instant messaging, and a Short Message Service (SMS).

The display device 210 may include suitable logic, circuitry, and interfaces that may be configured to display the recommendation associated with the food item. The display device 210 may be a touch screen which may enable a user (e.g., the user 110) to provide a user-input via the display device 210. The touch screen may be at least one of a resistive touch screen, a capacitive touch screen, or a thermal touch screen. The display device 210 may be realized through several known technologies such as, but not limited to, at least one of a Liquid Crystal Display (LCD) display, a Light Emitting Diode (LED) display, a plasma display, or an Organic LED (OLED) display technology, or other display devices. In accordance with an embodiment, the display device 210 may refer to a display screen of a head mounted device (HMD), a smart-glass device, a see-through display, a projection-based display, an electro-chromic display, or a transparent display.

The database of food items 212 may include the food items that may cause food allergies to a user (such as, the user 110 of FIG. 1). Further, the database of food items 212 may include the record of each food items that the user 110 may have consumed and faced food allergies. In an example, the database of food items may include, an identification (ID), the name, a date of consumption of the food item, a binary safe/unsafe value, and a one or more medications for each food item. Further, the database of food items 212 may be updated to include a new food item or to remove an existing food item. Various operations of the circuitry 202 are described further, for example, in FIGS. 3A, 3B, 4A, 4B, 5, and 6.

FIGS. 3A and 3B are diagrams that illustrates an exemplary scenario for generation of personalized recommendations to ensure safe consumption of foods items, in accordance with an embodiment of the disclosure. FIGS. 3A and 3B are explained in conjunction with elements from FIG. 1 and FIG. 2. With reference to FIGS. 3A and 3B, there is shown an exemplary scenario 300. The exemplary scenario 300 includes an electronic device 302 with a user interface (UI) 304A, a first UI element 306, a second UI element 308, a database of food items 312, a user interface (UI) 304B, a third UI element 314, a fourth UI element 316. The exemplary scenario 300 further includes an intermediate operation 310. A set of operations associated with the exemplary scenario is described herein.

During operation, the electronic device 302 may be configured to collect information associated with the user 110 of the electronic device 302. The collected information may be related to the user 110 and/or the food item 112 that the user 110 may intend to consume. As shown in FIG. 3, for example, the user 110 may intend to consume a chicken burger. The circuitry 202 may be configured to determine the food item 112 that the user 110 may intend to consume based on the collected information.

In an embodiment, the electronic device 302 may be further configured to control the display to render the user interface (UI) 304A that includes one or more options to query a database (such as the database 106 of FIG. 1). The circuitry 202 may be further configured to receive an input from the user 110 via the UI 304A. The collected information may include the input. As shown in FIG. 3A, the user 110 provides an input via the first UI element 306. The input includes a name of the food item 112, i.e., chicken burger.

In an embodiment, the input may include at least one of an audio that describes the food item 112, a text that includes a name of the food item 112, an image of the food item 112, or a video of the food item 112. For example, the UI 304A may include an option which when selected may allow the user 110 to input a voice description of the food item 112. The audio may include the voice description. Alternatively, the UI 304A may include an option to upload a prerecorded audio that describes the food item 112. The electronic device 302 may convert the audio to text and may determine the food item 112 from the text. The UI 304A may also include options for the user 110 to capture and upload an image of the food item 112 or to retrieve the image of the food item 112 from sources such as the Internet, a disk storage, or a cloud storage. The electronic device 302 may input the image to a machine learning (ML) model. The ML model may be trained to determine the food item 112 that the user 110 intends to consume based on the input image.

In an embodiment, the input may include a name of a store or a name of a restaurant, and the collected information may include a list of food items in a food menu of the store or the restaurant. In case the user 110 is unsure on whether it is safe to consume the food item 112, the name of the store or restaurant may be provided via the second UI element 308. For example, the user 110 may type the name of the store or restaurant via the second UI element 308. Alternatively, the electronic device 302 may determine a location of the electronic device 302 and may determine the name of the store or the restaurant at the determined location. Thereafter, the electronic device 302 may retrieve a list of food items from a food menu of the store or the restaurant, an online portal, and the like. The list of food items may include food items that the store or the restaurant may offer to its customers.

The food item 112 may be determined from the list of food items based on historical user data associated with food orders of the user 110. For example, the electronic device 302 may store information such as the name of the store or the restaurant, a list of names of the food items that the user 110 typically consumes at the store or the restaurant, or a list of dates of consumption of such food items. The stored information may be considered as the historical user data associated with the food orders. The electronic device 302 may determine the food item that the user 110 may have consumed recently based on the historical user data in the database 106. Alternatively, the food item that the user 110 may have consumed for a maximum number of times may be selected from the historical user data. The selected food item may be the food item 112.

At 310, the database 106 may be queried. The electronic device 302 may query the database 106 to determine whether the database 106 includes a record corresponding to the determined food item 112. If the record exists, then it may indicate that the user 110 may have experienced a food allergy or a food intolerance upon consumption of the determined food item 112 in the past. For example, the user 110 may have consumed pasta on a certain day and may have experienced allergic reactions due to gluten in the pasta. In past, the electronic device 302 may have collected, via a user input, the name of the food item that caused the allergic reactions, the date of consumption of the food item, the allergic reactions experienced by the user 110, the severity of the allergic reactions, and the like. The electronic device 302 may have stored the data associated with the food item in the database 106 as a record.

When the database 106 is queried, the electronic device 302 may search the database 106 to check whether the record for the determined food item 112 exits. As shown in FIG. 3A, for example, the electronic device 302 may search for the food item 112 that is, fried chicken burger in the database “Natalie_Food_Db” using a query “Search>Natalie_Food_Db”. The database 106 may include records associated with a plurality of food items. As shown in FIG. 3B, for example, the database of food items 312 may include a table with columns such as an identifier (ID), a name of the food item, a severity of reaction on a scale of one to ten, a date of consumption, a binary value that indicates whether the food item is safe or unsafe to consume (where zero may indicate that the food item is safe to consume and one may indicate that the food item is unsafe to consume), and a list of medications that may be required to treat the allergic reactions or digestive issues (associated with a food intolerance) caused by the food item. The record associated with the food item 112 may be present in the database of food items 312.

The electronic device 302 may generate a recommendation associated with the food item 112 based on whether the database 106 includes the record. The recommendation may indicate whether the food item 112 is safe for the user 110 to consume in light of the food allergies or the food intolerances. As shown in FIG. 3B, for example, the record associated with the food item 112 is present in the database of food items 312. The ID of the food item 112 is “25”, the name of the food item 112 is fried chicken burger, the severity of reaction of the food item 112 is 8 out of 10, the date of consumption of the food item 112 is 14th of August 2022, the binary safe/unsafe value is one (that indicates that the food item 112 is unsafe to consume), and a medication for the food allergy associated with the food item 112 is “Epinephrine”. The recommendation may include a prompt that may suggest the user 110 to not consume the food item 112. The circuitry 202 may control the display such as the display device 210 of the electronic device 302 to render the recommendation. As shown in FIG. 3B, for example, the third UI element 314 of the UI 304B may include a recommendation for the user 110 to not have the food item 112 as the user 110 had allergic reactions last time on 14 Aug. 2022 due to consumption of the fried chicken burger. The recommendation may include a suggestion for the user 110 to take Epinephrine to alleviate reactions associated with the food allergy in case the user 110 has decided to consume the fried chicken burger.

In an embodiment, the electronic device 302 may be further configured to query the database 106 to determine the preventive medication for the food allergy associated with the food item 112. The preventive medication may be determined based on a determination that the database 106 includes the record corresponding to the food item 112. The circuitry 202 may be further configured to control the display (such as the display device 210 of FIG. 2) to further render a notification that prompts the user 110 to check whether the user 110 has access to the medication before the user 110 decides to consume the food item 112. The circuitry 202 may be further configured to receive a user response to the notification. The recommendation may be generated further based on the user response. As an example, the electronic device 302 may recommend the user 110 to carry Epinephrine. Specifically, the electronic device 302 may render a notification that may prompt the user 110 to carry the preventative medication “Epinephrine”. The notification may include a text such as “Do you have the preventative medication Epinephrine with you?”. Further, UI elements with options such as “yes” and “no” may be rendered on the display of the electronic device 302. In case the user 110 has the preventative medication “Epinephrine”, the user response may include a selection of ‘yes’ option. In case the user 110 does not have the preventative medication “Epinephrine”, the user response may include a selection of a ‘no’ option. The electronic device 302 may not recommend the food item 112 for consumption based on the received user response. Other examples of the user response may include, but are not limited to, an audio about whether or not the user 110 has the preventative medication, a text that includes a yes or no response, an image of the preventative medication carried by the user 110, or a video of the preventative medication carried by the user 110.

In an embodiment, the electronic device 302 may be configured to determine one or more ingredients of the determined food item 112 as a cause of an acid reflux condition (i.e., a type of food intolerance). The one or more ingredients may be determined based on analysis of a dataset of food ingredients and allergens. The electronic device 302 may be configured to query a database such as the database 106 to determine whether the database 106 includes at least one record associated with the acid reflux condition. The recommendation may be generated further based on the determination that the database 106 includes at least one record associated with the acid reflux condition.

The backward flow of stomach acid into the tube that links the neck to the stomach is known as acid reflux. Often, one or more ingredients such as tomatoes, dairy products, carbonate beverages, and the like cause the acid reflux condition. Such ingredients of the food item 112 may have to be determined in a food item to ascertain if it is safe for the user 110 to consume the food item. Each ingredient may be searched in the database 106. In case any of the one or more ingredients is part of at least one record associated with the acid reflux condition, the corresponding food item may not be recommended to the user 110 for consumption. For example, in case tomatoes are present in the food item 112, that is, tomatoes in the fried chicken burger are found to cause acid reflux condition to the user 110, the food item 112 (i.e., the fried chicken burger) may not be recommended to the user 110. In some cases, the electronic device 302 may recommend one or more food ingredients such as nuts, broccoli, eggplant, or other suitable alternatives to include in a preparation of the food item 112 so as to balance an overall acidity level of the food item 112. For example, in case the food item 112 such as fried chicken burger contains tomatoes that lead to the acid reflux condition, then nuts such as almonds, cashews, chestnuts, hazelnuts, or walnuts may be recommended to the user 110. The user 110 may consume the nuts along with the food item 112. The combination of the recommended nuts and the food item 112 may balance the overall acidity level and may be safe for consumption.

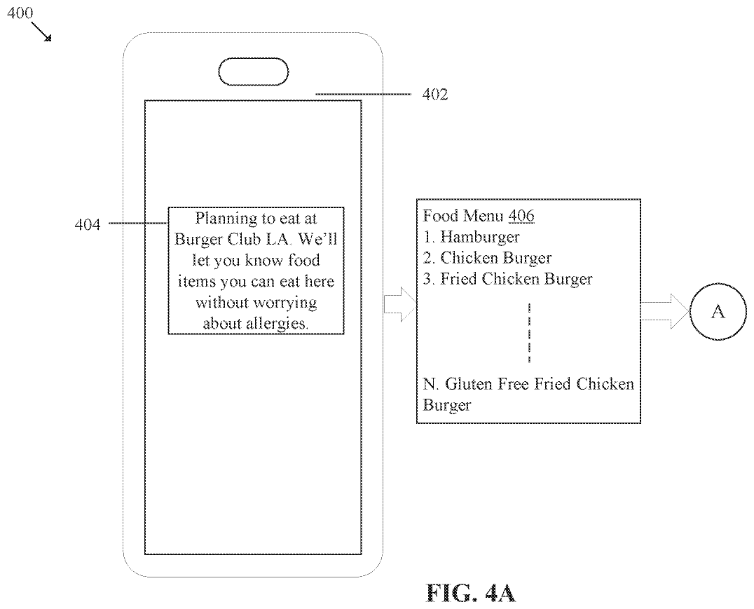

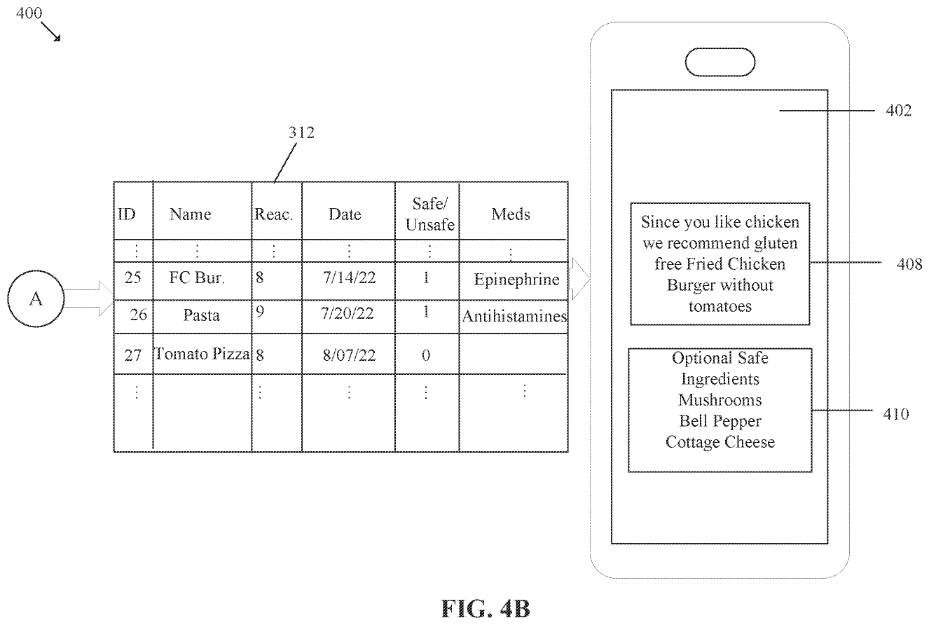

FIGS. 4A and 4B are diagrams that illustrate an exemplary scenario for generation of personalized recommendations to ensure safe consumption of foods items based on user's location, in accordance with an embodiment of the disclosure. FIGS. 4A and 4B are diagrams explained in conjunction with elements from FIG. 1, FIG. 2, FIG. 3A, and FIG. 3B. With reference to FIGS. 4A and 4B, there is shown an exemplary scenario 400. The exemplary scenario 400 may include the electronic device 302 with a user interface (UI) 402, a first UI element 404, a food menu 406, the database of food items 312, a second UI element 408, and a third UI element 410. The food menu 406 of the restaurant “Burger Club LA” may include N number of food items such as hamburger, chicken burger, fried chicken burger, and gluten free fried chicken burger. A set of operations associated with the exemplary scenario is described herein.

During operation, the circuitry 202 may determine a location of the electronic device 302 and may retrieve a list of food items in the food menu 406 of a store or a restaurant that corresponds to the determined location. The circuitry 202 may query a database such as the database 106 to determine that the database includes one or more records corresponding to one or more food items of the list of food items. The circuitry 202 may filter the list of food items based on a removal of the one or more food items. The food item that the user 110 should consume may be determined from the filtered list of food items.

As shown in FIG. 4A, the electronic device 302 may determine a location of the user 110 (or the electronic device 302). The first UI element 404 of the user interface (UI) 402 may provide the determined location of the electronic device 302 as “Burger Club LA”. The list of food items in the food menu 406 of Burger Club LA may be retrieved. Each of the list of food items in the food menu 406 of Burger Club LA may be searched for in the database of food items 312. If one or more records of the corresponding food item exists, then the corresponding food item may be removed from the list of food items to obtain the filtered list of food items. The filtered list of food items may include food items that may be safe for the user 110 to consume (i.e., safe enough to not cause any food allergy upon consumption). The filtered list of food items may be recommended to the user 110. As shown in FIG. 4B, for example, the gluten free fried chicken burger may not be in the database of food items 312. Thus, the second UI element 408 may include a recommendation for the user 110 to have the gluten free fried chicken burger.

In accordance with an embodiment, the electronic device 302 may query the database of the food items 312 for one or more ingredients of the food item 112 that the user 110 may be allergic to. As shown in FIG. 4B, the user 110 may be allergic or intolerant to tomatoes. The second UI element 408 may include a recommendation for the user 110 to have gluten free fried chicken burger without tomatoes. Further, the electronic device 302 may recommend one or more optional ingredients that the user 110 may consume instead of some of the ingredients of the food item 112 that the user 110 may be allergic to. As shown, for example, the third UI element 410 may include a recommendation of optional safe ingredients such as mushrooms, bell peppers, and cottage cheese slices. The user 110 may have to request the restaurant “Burger Club LA” to replace tomatoes with one or more of the recommended safe ingredients in the food item 112.

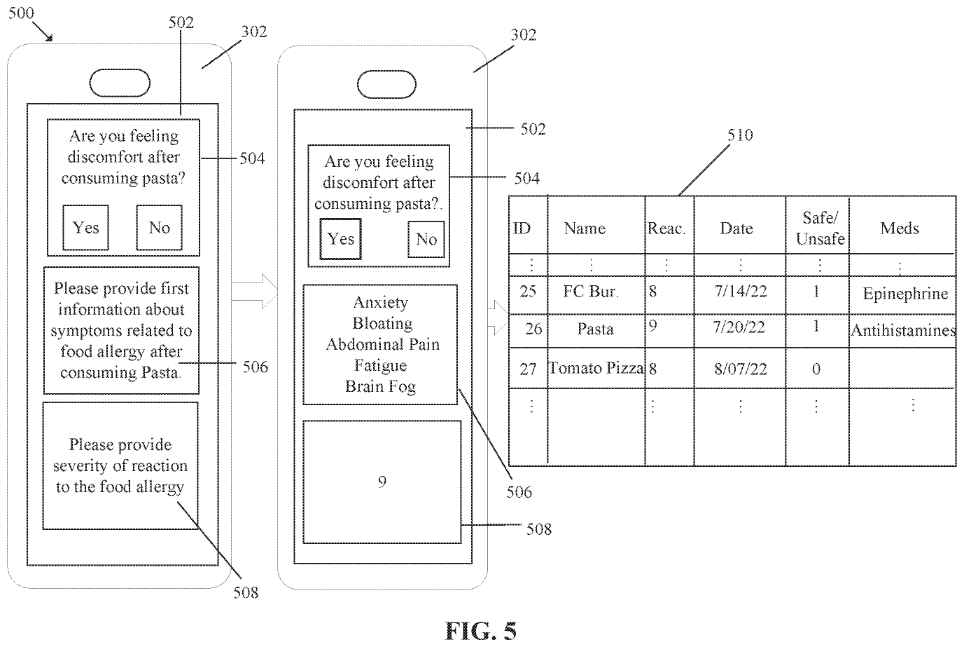

FIG. 5 is a diagram that illustrates an exemplary scenario for collection of health information associated with a user of an electronic device, in accordance with an embodiment of the disclosure. FIG. 5 is a diagram explained in conjunction with elements from FIG. 1, FIG. 2, FIG. 3A, FIG. 3B, FIG. 4A, and FIG. 4B. With reference to FIG. 5, there is shown an exemplary scenario 500. The exemplary scenario 500 may include the electronic device 302 a user interface (UI) 502, a first UI element 504, a second UI element 506, a third UI element 508, and the database of food items 510. The food menu 406 of the restaurant “Burger Club LA” may include N number of food items, such as hamburger, chicken burger, fried chicken burger, and gluten free fried chicken burger. A set of operations associated with the exemplary scenario is described herein.

During operation, the circuitry 202 may be configured to collect health information associated with the user 110. The health information may include first information about symptoms associated with one or more food allergies or one or more food intolerances that the user 110 experiences after the user 110 consumes a food item, and second information about a severity of reaction to such food allergies or such intolerances that the user experiences. The circuitry 202 may be configured to update the database 106 to include the food item and the collected health information.

As shown, for example, the electronic device 302 may query the user 110 to determine whether the user 110 is experiencing any discomfort after a meal. The first UI element 504 may display a question, “Are you feeling discomfort after consuming pasta?”. The first UI element 504 may provide an option “yes” and an option “no”. If the option “yes” is selected, then the second UI element 506 may prompt the user 110 to provide first information about symptoms related to the food allergy. Through a user input, the first information may be provided by the user 110. As an example, the user input may include symptoms such as anxiety, bloating, abdominal pain, fatigue, and brain fog. The third UI element 508 may prompt the user 110 to provide a severity of reaction to the food allergy on the scale of one to ten. Through another user input, the severity of reaction may be provided by the user 110. As an example, the input may provide the severity of reaction as nine (9/10). The database of the food items 510 may be updated to include the symptoms that the user 110 experienced after the user 110 consumed the food item and the severity of reaction to the food allergy.

In an embodiment, the collected health information may correspond to a past instance of a consumption of the food item, and the recommendation may be generated based on whether the severity of reaction corresponding to the past instance is a moderate reaction or a severe reaction. Based on the symptoms in the foregoing example, the severity of reaction may be categorized as a moderate reaction or a severe reaction.

In an embodiment, the severity of reaction is defined on a scale of one to ten. If the severity of reaction is below six, then the severity of reaction may be considered as a moderate reaction. If the severity of reaction is greater than or equal to six, then the severity of reaction may be considered as the severe reaction. In case the severity of reaction corresponding to the past instance is a moderate reaction, the corresponding food item may be recommended to the user 110 for consumption with due precautions. However, if the severity of reaction corresponding to the past instance is a severe reaction, then the corresponding food item may be marked as unsafe to consume for the user 110 and may not be recommended to the user 110.

As shown in FIG. 5, for example, when the user 110 consumed pasta on 20 Aug. 2022, the user 110 faced one or more food allergies and the severity of reaction was 9. Thus, the user 110 may not be recommended pasta in future. However, if the severity of reaction drops with a pre-meal medication, then the corresponding food item may be recommended to the user 110 with due precautions.

In an embodiment, the circuitry 202 may be configured to receive a user input that includes the health information. The health information may include data related to the health of the user 110 over a period of time. For example, the user 110 may be prohibited from consuming certain food items that pose a risk to the user's health. For example, in case the user 110 is a diabetics patient, then the recommendation for a food item may consider an amount of sugar in the food item.

In an embodiment, the circuitry 202 may be configured to monitor a set of health parameters of the user 110 via one or more sensors. At a first time-instant, the circuitry 202 may detect an abnormal change in the set of health parameters within a duration in which the set of health parameters is monitored. In an embodiment, the set of health parameters may include least one of an oxygen saturation level, a heartbeat or pulse rate, a blood pressure, and a respiration rate, the like. An oximeter may measure the oxygen saturation level for the user 110. The circuitry 202 may monitor the oxygen saturation levels over a period. In case the oxygen saturation level for the user 110 falls below a threshold (such as 95%), then the abnormal change may be detected. In an embodiment, the health information may be collected based on the detection of the abnormal change in the set of health parameters. For example, when the user 110 consumes the food item 112, then the heartbeat for the user 110 may be reach 120 beats per minute (i.e., higher than a normal range of 60-80 beats per minute). The measured heartbeat of 120 beats per minute may be treated as abnormal and may be due to an allergic reaction of the user's body upon a consumption of the food item 112. The health information may be collected so that the user 110 is not recommended the food item 112 in future based on a severity of the abnormal change.

In an embodiment, the circuitry 202 may be configured to acquire a dataset of food ingredients and allergens. The circuitry 202 may be further configured to construct a training dataset that includes training records corresponding to the food items of the database 106. The dataset may be constructed based on the acquired dataset and the database of the food items. The circuitry 202 may be further configured to train a machine learning model on a task of binary classification, based on the training dataset.

Ingredients that cause the food allergy are typically referred to as allergens. For example, one or more food ingredients of the food item 112 may be chicken, gluten, eggs, parsley leaves, onions garlic, paprika, salt and pepper. The gluten in the flour may cause a food allergy to the user 110. Thus, gluten may be an allergen of the food item 112. Similarly, one or more food ingredients and respective allergens may be extracted from the database of food items to form the training dataset. The training dataset may be used for training the machine learning model on a task of binary classification. The task of binary classification may be to determine whether the determined food item is safe or unsafe for the user 110 to consume. In case at least one ingredient of the one or more food ingredients of a food item is an allergen, then the determined food item may be labeled as unsafe for consumption by the machine learning model.

In an embodiment, the training dataset may include at least one of identifiers of the food items that correspond to records of the database, food ingredients associated with the food items, allergens in the food items, severity of reactions that the user may have experienced due to different allergens, health risk indications corresponding to the allergens, or class labels to indicate whether the food ingredients are safe or unsafe to consume in light of the allergens. Herein, the identifiers may be an ID of the food item in in the database. The food ingredients associated with a food item may be items that may be used to prepare the food item. The severity of reaction that the user experiences due to each of the allergen may be provided on a scale of one to ten, where ten may indicate a severe reaction and one may indicate a mild reaction. The health risk indication corresponding to each of the allergen may indicate a health risk posed by the allergen. Examples of the health risk may include, but are not limited to, a difficult or noisy breathing, a swelling of the tongue, a swelling or tightness in the throat, a condition such as anaphylaxis, and the like. The class labels may specify whether the food ingredient is safe to consume or not. For example, food items prepared with all-purpose flour may be unsafe to consume due to presence of gluten in the flour.

In an embodiment, the circuitry 202 may be configured to generate an input feature for the determined food item and feed the input feature to the trained machine learning (ML) model. The circuitry 202 may be further configured to extract an output of the trained ML model for the fed input feature. The recommendation may be generated further based on the output. The output may indicate an allergen in the food item, a severity of reaction to the allergen, a health risk indication corresponding to the allergen and a recommendation on whether the food item is safe or unsafe to consume in light of the allergen. For example, the input feature of a food item may include wheat, fish oil, eggs, parsley, and pepper. The trained ML model may output a label that may mark the food item as unsafe to consume for the user 110. The label may be used to recommend the user 110 to skip fish oil (i.e., allergen) in case the user 110 wishes to consume the determined food item.

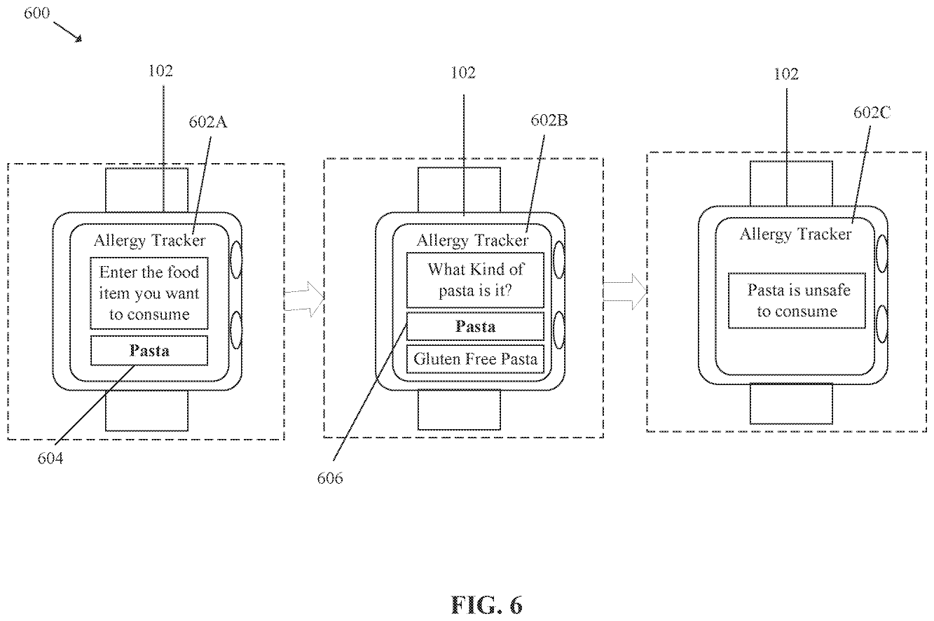

FIG. 6 is a diagram that illustrates an exemplary scenario for generation of personalized recommendations to ensure safe consumption of foods items, in accordance with an embodiment of the disclosure. FIG. 6 is a diagram explained in conjunction with elements from FIG. 1, FIG. 2, FIG. 3A, FIG. 3B, FIG. 4A, FIG. 4B, and FIG. 5. With reference to FIG. 6, there is shown an exemplary scenario 600. The exemplary scenario 600 may include the electronic device 102, a user interface (UI) 602A, a UI 602B, a UI 602C, a first UI element 604, and a second UI element 606. A set of operations associated with the exemplary scenario is described herein.

In operation, the electronic device 102 may request, via the UI 602A, the user 110 of the electronic device 102 to provide the food item that the user 110 wishes to consume. Through a user input from the user 110 via the UI 602A, the name of the food item may be provided. For example, the name of the food item may be pasta. Thereafter, the electronic device 102 may request the user 110 of the electronic device 102 to provide a type of pasta that the user 110 wishes to consume. Through another user input via the UI 602B, the type of pasta may be provided as a gluten free pasta. The electronic device 102 may query the database 106 based on the name and the type of pasta and may recommend to the user 110 to not consume the pasta via the UI 602C.

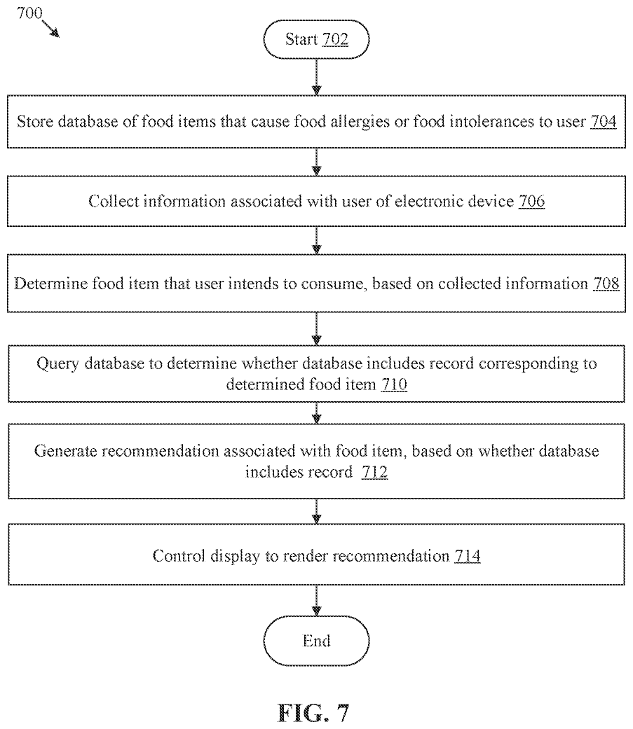

FIG. 7 is a flowchart that illustrates operations of an exemplary method for generation of personalized recommendations to ensure safe consumption of foods items, in accordance with an embodiment of the disclosure. FIG. 7 is a diagram explained in conjunction with elements from FIG. 1, FIG. 2, FIG. 3A, FIG. 3B, FIG. 4A, FIG. 4B, FIG. 5, and FIG. 6. With reference to FIG. 7, there is shown a flowchart 700. The flowchart 700 may include operations from 702 to 714 and may be implemented by the electronic device 102 of FIG. 1 or by the circuitry 202 of FIG. 2. The flowchart 700 may start at 702 and proceed to 704.

At 704, the database of food items that causes food allergies or food intolerances to the user may be stored. The circuitry 202 may be configured to store the database of food items 212 that cause food allergies to the user (such as, the user 110 of FIG. 1). Details related to the database of food items are provided, for example, in FIG. 2.

At 706, the information associated with the user 110 of the electronic device 102 may be collected. The circuitry 202 may be configured to collect information associated with the user 110 of the electronic device 102. The collected information may relate to the food item 112 that the user 110 may intend to consume. In an example, the collected information may be the name of the food item, the type of food item, ingredients of the food item, and the like. Details related to the collection of information are provided, for example, in FIG. 3A.

At 708, the food item 112 that the user 110 intends to consume may be determined based on the collected information. The circuitry 202 may be configured to determine the food item 112 that the user 110 intends to consume, based on the collected information. For example, if the collected information is the image of the food item 112 then the image may be provided as the input to the trained ML model that may identify the name, type, ingredients, and the like of the food item 112. Details related to determining the food item 112 are provided, for example, in FIG. 3A.

At 710, the database 106 may be queried to determine whether the database 106 includes the record corresponding to the determined food item 112. The circuitry 202 may be configured to query the database 106 to determine whether the database 106 includes the record corresponding to the determined food item 112. Herein, the determined food item 112 may be searched for in the database such as, the database of food items 312 to determine whether the record of the determined food item 112 exists or not. Details related to determining whether the database 106 includes the record corresponding to the determined food item 112 are provided, for example, in FIG. 3B.

At 712, the recommendation associated with the food item 112 may be generated based on whether the database 106 includes the record. The circuitry 202 may be configured to generate the recommendation associated with the food item 112, based on whether the database 106 includes the record. The recommendation may indicate whether the food item 112 is safe for the user 110 to consume in light of the food allergies. Details related to the recommendation associated with the food item 112 are provided, for example, in FIG. 3B.

At 714, the display such as, the display device 210 may be controlled to render the recommendation. The circuitry 202 may be configured to control the display to render the recommendation. The rendered recommendation may also include preventive medications that the user 110 may have consume to in order to combat food allergy associated with the determined food item 112. Further, the rendered recommendation may include food ingredients that the user 110 may be allergic to and may ask the restaurant or the store to omit them when preparing the determined food item. The rendered recommendation may include optional ingredients that the user 110 may consume. Details related to the rendering of the recommendation are provided, for example, in FIG. 3B. Control may pass to end.

Although the flowchart 700 is illustrated as discrete operations, such as, 704, 706, 708, 710, 712, and 714, the disclosure is not so limited. Accordingly, in certain embodiments, such discrete operations may be further divided into additional operations, combined into fewer operations, or eliminated, depending on the implementation without detracting from the essence of the disclosed embodiments.

Various embodiments of the disclosure may provide a non-transitory computer-readable medium and/or storage medium having stored thereon, computer-executable instructions executable by a machine and/or a computer to operate an electronic device (for example, the electronic device 102 of FIG. 1). Such instructions may cause the electronic device 102 to perform operations that may include collecting information associated with a user (such as, the user 110 of FIG. 1) of the electronic device. The operations may further include determining a food item (such as, the food item 112 of FIG. 3A) that the user intends to consume, based on the collected information. The operations may further include querying a database (such as, the database of food items 312 of FIG. 3) to determine whether the database includes a record corresponding to the determined food item. The database may be for food items that cause food allergies to a user (such as, the user 110 of FIG. 1). The operations may further include generating a recommendation associated with the food item, based on whether the database includes the record. The recommendation may indicate whether the food item is safe for the user to consume in light of the food allergies. The operations may further include controlling a display (such as, the display device 210 of FIG. 2) of the electronic device to render the recommendation.

Exemplary aspects of the disclosure may provide an electronic device (such as, the electronic device 102 of FIG. 1) that includes, a display (such as, the display device 210 of FIG. 2), a memory (such as, the memory 204 of FIG. 2) configured to store a database of food items (such as, the database of food items 212 of FIG. 2) that cause food allergies to a user (such as, the user 110 of FIG. 1), and the circuitry (such as, the circuitry 202). The circuitry 202 may be configured to collect information associated with a user (such as, the user 110 of FIG. 1) of the electronic device. The circuitry 202 may be configured to determine a food item (such as, the food item 112 of FIG. 3A) that the user intends to consume, based on the collected information. The circuitry 202 may be configured to query a database (such as, the database of food items 312 of FIG. 3) to determine whether the database includes a record corresponding to the determined food item. The database may be for food items that cause food allergies to a user (such as, the user 110 of FIG. 1). The circuitry 202 may be configured to generate a recommendation associated with the food item, based on whether the database includes the record. The recommendation may indicate whether the food item is safe for the user to consume in light of the food allergies. The circuitry 202 may be configured to control a display (such as, the display device 210 of FIG. 2) of the electronic device to render the recommendation.

In an embodiment, the circuitry 202 may further configured to control the display (such as, the display device 210 of FIG. 2) to render the user interface (UI) 304A that may include one or more options to query the database 106. The circuitry 202 may further configured to receive an input from the user 110 via the UI 304A wherein the collected information includes the input.

In an embodiment, the input may include at least one of an audio about the food item 112, a text that includes a name of the food item 112, an image of the food item 112, or a video of the food item 112.

In an embodiment, the input may include a name of a store or a name of a restaurant, and the collected information includes a list of food items in the food menu 406 of the store or the restaurant.

In an embodiment, the food item 112 may be determined from the list of food items based on historical user data associated with food orders of the user 110.

In an embodiment, the circuitry 202 may be further configured to determine a location of the electronic device 102. The circuitry 202 may be further configured to retrieve a list of food items in the food menu 406 of a store or a restaurant that corresponds to the determined location. The circuitry 202 may be further configured to query the database (such as, the database of food item 312 of FIG. 4B) to determine that the database includes one or more records corresponding to one or more food items of the list of food items. The circuitry 202 may be further configured to filter the list of food items based on a removal of the one or more food items, wherein the food item is determined from the filtered list of food items.

In an embodiment, the circuitry 202 may be further configured to collect health information associated with the user 110. The health information may include first information about symptoms associated with one or more food allergies that the user 110 experiences after the user 110 consumes the food item 112, and second information about a severity of reaction to the one or more food allergies that the user experiences. The circuitry 202 may be further configured to update the database 106 to include the food item 112 and the collected health information.

In an embodiment, the collected health information may correspond to a past instance of a consumption of the food item 112, and the recommendation may be generated further based on whether the severity of reaction corresponding to the past instance is a moderate reaction or a severe reaction.

In an embodiment, the circuitry 202 may be further configured to receive a user input that includes the health information.

In an embodiment, the circuitry 202 may be further configured to monitor a set of health parameters of the user 110 via one or more sensors. The circuitry 202 may be further configured to detect an abnormal change in the set of health parameters at a first time-instant in a duration in which the set of health parameters is monitored.

In an embodiment, the health information may be collected based on the detection of the abnormal change in the set of health parameters.

In an embodiment, the circuitry 202 may be further configured to query the database 106 to determine a preventive medication for a food allergy associated with the food item 112. The preventive medication may be determined based on the determination that the database 106 includes the record corresponding to the food item 112. The circuitry 202 may be further configured to control the display (such as, the display device 210 of FIG. 2) to further render a notification that prompts the user 110 to check whether the user 110 has access to the preventative medication before the user 110 decides to consume the food item 112. The circuitry 202 may be further configured to receive a user response to the notification. The recommendation may be generated further based on the user response.

In an embodiment, the circuitry 202 may be further configured to determine one or more ingredients of the determined food item 112 as a cause of an acid reflux condition, wherein the one or more ingredients are determined based on an analysis of a dataset of food ingredients and allergens. The circuitry 202 may be further configured to query the database 106 to determine whether the database 106 includes at least one record associated with the acid reflux condition. The recommendation may be generated further based on the determination that the database 106 includes the at least one record associated with the acid reflux condition.

In an embodiment, the circuitry 202 may be further configured to acquire a dataset of food ingredients and allergens. The circuitry 202 may be further configured to construct a training dataset that includes training records corresponding to the food items of the database 106. The dataset may be constructed based on the acquired dataset and the database of the food items 212. The circuitry 202 may be further configured to train a machine learning model on a task of binary classification, based on the training dataset.

In an embodiment, the training dataset may include at least one of: identifiers of the food items that correspond to records of the database 106, food ingredients associated with the food items, allergens in the food items, a severity of reaction that the user 110 experiences due to each of the allergens, a health risk indication corresponding to each of the allergens, or class labels to indicate whether the food ingredient is safe or unsafe to consume in light of the allergens.

In an embodiment, the circuitry 202 may be further configured to generate an input feature for the determined food item 112. The circuitry 202 may be further configured to feed the input feature to the trained machine learning model. The circuitry 202 may be further configured to extract an output of the trained machine learning model for the fed input feature. The recommendation may be generated further based on the output.

The present disclosure may also be implemented in a computer program product, which comprises all the features that enable the implementation of the methods described herein, and which when loaded in a computer system is able to carry out these methods. Computer program, in the present context, means any expression, in any language, code or notation, of a set of instructions intended to cause a system with information processing capability to perform a particular function either directly, or after either or both of the following: a) conversion to another language, code or notation; b) reproduction in a different material form.

While the present disclosure is described with reference to certain embodiments, it will be understood by those skilled in the art that various changes may be made, and equivalents may be substituted without departure from the scope of the present disclosure. In addition, many modifications may be made to adapt a particular situation or material to the teachings of the present disclosure without departure from its scope. Therefore, it is intended that the present disclosure is not limited to the embodiment disclosed, but that the present disclosure will include all embodiments that fall within the scope of the appended claims.

What is claimed is:

1. An electronic device, comprising:
a display;
a memory configured to store a database of food items that cause food allergies or food intolerances to a user; and
circuitry configured to:
collect information associated with a user of the electronic device;
determine a food item that the user intends to consume based on the collected information;
query the database to determine whether the database includes a record corresponding to the determined food item;
generate a recommendation associated with the food item based on whether the database includes the record,
wherein the recommendation indicates whether the food item is safe for the user to consume in light of the food allergies or the food intolerances; and
control the display to render the recommendation.

2. The electronic device according to claim 1, wherein the circuitry is further configured to:
control the display to render a user interface (UI) that includes one or more options to query the database; and
receive an input from the user via the UI, wherein the collected information includes the input.

3. The electronic device according to claim 2, wherein the input includes at least one of an audio about the food item, a text that includes a name of the food item, an image of the food item, or a video of the food item.

4. The electronic device according to claim 2, wherein the input includes a name of a store or a name of a restaurant, and the collected information includes a list of food items in a food menu of the store or the restaurant.

5. The electronic device according to claim 4, wherein the food item is determined from the list of list of food items based on historical user data associated with food orders of the user.

6. The electronic device according to claim 1, wherein the circuitry is further configured to:
determine a location of the electronic device;
retrieve a list of food items in a food menu of a store or a restaurant that corresponds to the determined location;
query the database to determine that the database includes one or more records corresponding to one or more food items of the list of food items; and
filter the list of food items based on a removal of the one or more food items, wherein the food item is determined from the filtered list of food items.

7. The electronic device according to claim 1, wherein the circuitry is further configured to:
collect health information associated with the user,
wherein the health information includes:
first information about symptoms associated with one or more food allergies or one or more food intolerances that the user experiences after the user consumes the food item, and
second information about a severity of reaction to the one or more food allergies or the one or more food intolerances; and
update the database to include the food item and the collected health information.

8. The electronic device according to claim 7, wherein the collected health information corresponds to a past instance of a consumption of the food item, and the recommendation is generated further based on whether the severity of reaction corresponding to the past instance is a moderate reaction or a severe reaction.

9. The electronic device according to claim 7, wherein the circuitry is further configured to receive a user input that includes the health information.

10. The electronic device according to claim 7, wherein the circuitry is further configured to:
monitor a set of health parameters of the user via one or more sensors; and
detect an abnormal change in the set of health parameters at a first time-instant in a duration in which the set of health parameters is monitored.

11. The electronic device according to claim 10, wherein the health information is collected based on the detection of the abnormal change in the set of health parameters.

12. The electronic device according to claim 1, wherein the circuitry is further configured to:
query the database to determine a preventive medication for a food allergy associated with the food item or a food intolerance associated with the food item,
wherein the preventive medication is determined based on the determination that the database includes the record corresponding to the food item;
control the display to further render a notification that prompts the user to check whether the user has access to the preventative medication before the user decides to consume the food item; and
receive a user response to the notification,
wherein the recommendation is generated further based on the user response.

13. The electronic device according to claim 1, wherein the circuitry is further configured to:
determine one or more ingredients of the determined food item as a cause of an acid reflux condition, wherein the one or more ingredients are determined based on an analysis of a dataset of food ingredients and allergens; and
query the database to determine whether the database includes at least one record associated with the acid reflux condition,
wherein the recommendation is generated further based on the determination that the database includes the at least one record associated with the acid reflux condition.

14. The electronic device according to claim 1, wherein the circuitry is further configured to:
acquire a dataset of food ingredients and allergens;
construct a training dataset that includes training records corresponding to the food items of the database,
wherein the dataset is constructed based on the acquired dataset and the database of the food items; and
train a machine learning model on a task of binary classification based on the training dataset.

15. The electronic device according to claim 14, wherein the training dataset includes at least one of:
identifiers of the food items that correspond to records of the database, food ingredients associated with the food items,
allergens in the food items,
a severity of reaction that the user experiences due to each of the allergens, a health risk indication corresponding to each of the allergens, or class labels to indicate whether the food ingredient is safe or unsafe to consume in light of the allergens.

16. The electronic device according to claim 14, wherein the circuitry is further configured to:
generate an input feature for the determined food item;
feed the input feature to the trained machine learning model; and
extract an output of the trained machine learning model for the fed input feature, wherein the recommendation is generated further based on the output.

17. A method, comprising:
in an electronic device that includes a display and a memory storing a database of food items that cause food allergies or food intolerances to a user:
collecting information associated with a user of the electronic device;
determining a food item that the user intends to consume, based on the collected information;
querying the database to determine whether the database includes a record corresponding to the determined food item;
generating a recommendation associated with the food item based on whether the database includes the record,
wherein the recommendation indicates whether the food item is safe for the user to consume in light of the food allergies or the food intolerances; and
controlling the display to render the recommendation.

18. The method according to claim 17, further comprising:
controlling the display to render a user interface (UI) that includes one or more options to query the database; and
receiving an input from the user via the UI, wherein the collected information includes the input.

19. The method according to claim 18, wherein the input includes at least one of an audio about the food item, a text that includes a name of the food item, an image of the food item, or a video of the food item.

20. A non-transitory computer-readable medium having stored thereon, computer-executable instructions that when executed by an electronic device, causes the electronic device to execute operations, the operations comprising:
collecting information associated with a user of the electronic device;
determining a food item that the user intends to consume, based on the collected information;
querying a database to determine whether the database includes a record corresponding to the determined food item,
wherein the database is for food items that cause food allergies or food intolerances to a user;
generating a recommendation associated with the food item based on whether the database includes the record,
wherein the recommendation indicates whether the food item is safe for the user to consume in light of the food allergies or the food intolerances; and
controlling a display of the electronic device to render the recommendation.
